(19) United States
(12) Patent Application Publication
(10) Pub. No.: US 2012/0141610 A1
(43) Pub. Date: Jun. 7, 2012

(54) Novel Topical Composition Of Sarracenia Purpurea (Pitcher Plant)

(76) Inventor: Brandie Gowey, Flagstaff, AZ (US)

(21) Appl. No.: 13/309,144

(22) Filed: Dec. 1, 2011

Related U.S. Application Data
(60) Provisional application No. 61/418,692, filed on Dec. 1, 2010.
(60) Provisional application No. 61/448,824, filed on Mar. 3, 2011.

(51) Int. Cl. A61K 36/185; A61P 35/00

(52) U.S. Cl. 424/725

(57) ABSTRACT

A composition having a pitcher plant component and a fatty acid component, wherein the pitcher plant component is an alcohol extract, the pitcher plant component further comprises a preservative component, the pitcher plant component is prepared through a soak and press technique and prepared through steam distillation, the composition of claim 1 being a topical or suppository formulation.

### CROSS REFERENCE

The present non-provisional patent application claims benefit to the earlier priority date of provisional patent application Ser. No. 61/418,692 (filed Dec. 1, 2010) and provisional patent application Ser. No. 61/448,824 (filed Mar. 3, 2011), the disclosure of which is incorporated in its entirety herein by reference.

### BACKGROUND OF THE INVENTION

Sarracenia purpurea (pitcher plant) has historically been used internally as an anti-viral. The first recorded uses were reported by Native Americans to treat small pox Lancet 80: 430-431 (1862). Other members of the Sarracenia genus have also exhibited potent anti-viral activities. For example, betulin, which is a component of Sarracenia flava extract, exhibited anti-viral activities against herpes zoster (Weckesser et al. Forsch Komplementmed. 2010 17(5): 271-3).

Members of the Sarracenia genus exhibit potent anti-cancer activities. Extracts of the roots and leaves of Sarracenia flava exhibited anti-cancer activity against lymphocytic leukemia and human epidermoid carcinoma of the nasopharynx (Miles and Kokpol). J. Pharm. Science. 1976 65(2): 284-5; Miles and Kokpol. J. Pharm. Science. 1974 63(4): 613-50). The lupeol and betulin present in the Sarracenia flava extract exhibited anti-tumor activity against melanoma and brain tumors (Biechele and Chien. Carcinogenesis. 2011 32(1): 120; Fuda et al. Int. J. Cancer. 1999 82(3):435-41; Mullauer et al. Anticancer Drugs. 2010 21(3): 215-27; and Csuk et al. Achive der Pharm. 2011 344(1): 37-49).

Sarracenia purpurea exhibits analgesic activities. For example, an injectable form of soluble salts from Sarracenia purpurea inhibit neuromuscular and neuralgic pain in patients (www.sarapin.com).

There are innumerable subspecies of Sarracenia purpurea, examples include Sarracenia purpurea subspecies purpurea and Sarracenia purpurea subspecies venosa. Horticulturalists and Master Gardeners who specialize in growing these species have all found that the species interbreed easily, making it nearly impossible to not have subspecies, a common example being S. purpurea subspecies purpurea (www.pitcherplant.com). Since species of Sarracenia genus can interbreed, genetic and phenotypic varieties of Sarracenia species are readily found in regions where the species co-habitat. This propensity for interbreeding keeps the species population diverse, lending to a tendency for the generation of concentrated and naturally selected constituent levels that may be valuable therapeutically.

The Sarracenia purpurea extract contains anthocyanins and their glucosides including pelargonidin, pelargonidin 3-glucoside, cyanidin, cyanidin 3,5-diglucoside, cyanidin 3-glucoside monoglucuronide, peonidin, delphinidin, malvidin, and quercetin (Sheridan and Griesbach, 2001 and The International Carnivorous Plant Society). Anthocyanins are the largest group of water-soluble pigments in the plant kingdom, and are the constituents that give plants their color, commonly known as “antioxidants”. Their stability depends on pH (Bao J et al. J. Agric Food Chem. 2005 53(6):2327-32). Anthocyanins exhibit several layers of pharmacological activity: primarily inducing programmed cell death in infected or cancerous cells (apoptosis) while reducing inflammation and inhibiting tumor cell angiogenesis (http://en.wikipedia.orq/wiki/anthocyanin). The Sarracenia purpurea extract also contains 1,4-napthoquinone derivatives including plumbagin, juglone, and menadione, which exhibit strong anti-oxidant and cytotoxic activities (The International Carnivorous Plant Society).

### SUMMARY

The present invention features a medically active topical formulation of the liquid extract of the pitcher plant (“present composition” or “composition”). It is surprisingly discovered that the present composition is effective against lesions or diseases on skin that manifest as a result of the deleterious effects of viruses, bacteria, and cancerous cells.

In some embodiments, a pitcher plant component of the present invention comprises an extract of the pitcher plant, e.g., a liquid extract (also called a tincture). In some embodiments, the pitcher plant extract is obtained by methods well known to one of ordinary skill in the art, for example methods disclosed herein.

In some embodiments, the present composition is formulated with a base that holds the tincture of Sarracenia purpurea (liquid extract of all plant constituents, a type of herbal extract of organic grain alcohol and distilled water, with some forumations using glycerin even though it is not a solvent); in suspension and at the right pH for the plant constituents to be active topically. In some embodiments, a gel that can be used in accordance with the present invention is one that is provided by Professional Compounding Pharmacies of America (PCCA), and is known as “versabase gel”. VersaBase® Gel PCCA part number is 30-3656 (BOM version-001), which can be purchased at PCCA, 9901 South Wilcrest Dr., Houston, Tex. 77099-5132, Ph: 800.331.2498, Fax: 832.295.1215. This gel in and of itself is designed as an intert base for compounding formulations, make by compoounding pharmacists, for topical preparations. Also, the liquid extract in and of itself is inactive topically and may cause pain (extract contains alcohol and as such, when used on an open wound may cause pain) as it is designed for oral use only. The present composition comprises about 0.1 to 25% of the pitcher plant liquid extract in the versabase gel base for topical applications on human tissues. Anthocyanins as are listed in [0006] of this document may be added to standardize specific anthocyanin formulation, such as adding 0.01-2.0% delphinidin or cyanidin to further potentize the activity of the present composition.

One of the uniqueness of the present invention is topical application of the Sarracenia purpurea herb as well as its use in fatty acid emulsion suspensions (water bases with the tincture formulated as particles that suspend in fatty bases such as may be used in creams or lip balms) or versabase gel preparations. In some embodiments, additional anti-viral herbs may be added to this formula base such as Echinacea purpurea, Echinacea angustifolia, Lobelia, Lomatium spp, Usnea, Betulin, Lonicera spp, Populus spp, Drosera spp, Sarracenia spp, Nepthenes spp, or St. John's Wort or other bases; polymers, coplymers, emulsifiers, binders, and tissue-soothers such as Vitamin A, Vitamin E, cocoa butter, oils/creams, glucononolactone, DMSO, sodium hyaluronate, hyaluronic acid, lecithin, glycerin, water, ammonium acryloyldimethyltaurate/VP copolymer, aloe vera, edetate disodium, allantoin and methylchloroisothiazolinone/methylisothiazolinone, pelargonidin, pelargonidin 3-glucoside, cyanidin, cyanidin 3,5-diglucoside, cyanidine 3-glucoside monoglucuronide, peonidin, delphinidin, malvidin, quercetin, related anthocyanins and their glucosides; and preservatives such as sodium benozate.

Additional therapeutically active compounds can be added to the Sarracenia purpurea extract during the formulation process, for example, extracts (ECGC generally) of green tea, which have been shown to be effective in the treatment of external anogenital warts and is an FDA-approved treatment for cervical dysplasia/HPV (Tzellos et al. J. Eur. Acad. Dermatol. Venereol. 2011 25(3): 345-53).

In some embodiments, suppositories can also be formulated for use in vaginal applications in fatty acid bases that also hold the liquid extract in suspension and at the appropriate pH. These suppositories hold up to about 6% of Sarracenia purpurea liquid extract; however, they are not generally effective unless they are formulated such that the water-extracted constituents of S. purpurea (i.e., the anthocyanins) are fully emulsed with bases such as lecithin to be held in suspension in a fat.

The Formula Worksheet at the end of this non-provisional application provides non-limiting examples of the formulation procedures for making the topical composition and the suppository.

In some embodiments, a method of treatment using suppository formulation, inserted nightly for duration of treatment.

As used herein, the term “about” refers to plus or minus 10% of the referenced number.

### DESCRIPTION OF PREFERRED EMBODIMENTS

### Methods for Extraction

The Soak and Press Technique is the traditional method for preparing liquid extracts of herbs. To do this method, first the pitchers of the Sarracenia purpurea plant are collected, preferably from plants grown in the absence of pesticides. The Sarracenia purpurea leaves are cut lengthwise, and all detritus and dirt is manually removed. The plant usually contains 60% or more water by weight. The Sarracenia purpurea leaves are then prepared using the standard soak and press technique outlined by U.S. Pharmacopeia (Green, James. The Herbal Medicine Makers Handbook. The Crossing Press, 2000), although the coldfinger distillation method is preferred (see 0035).

The Sarracenia purpurea leaves can be used in their whole form, chopped or pureed. The solvent, which is also called the menstrum, is added to the Sarracenia purpurea leaves. A solvent consisting anywhere from 30/65 to 60/30 of 190 proof alcohol (for example Everclear or organic sugarcane or grain alcohol) and distilled water is efficient in preparing a Sarracenia purpurea extract. The solvent ratio can be adjusted depending on the inherent water content of the Sarracenia purpurea, which will change each year as a consequence of environmental factors.

It is important to note that glycerin, while commonly listed in herbal liquid extractions, is NOT used as a menstrum in the extraction process; it may be added at the end of the alcohol/water extraction as a preservative and binder of the solution; it does not actively extract constituents (according to the experts at Eden labs, www.edenlabs.org).

For example, if the plant experienced a very rainy season, it will have a higher water content, and a 60:40 ratio of solvent can be used for extraction. If the plant experienced a drier season, its water content will be lower, and a 65:35 ratio of solvent can be used for extraction. The ratio can be further adjusted to improve the extraction process if necessary: the alcohol can range from 5-90%; distilled water can range from 5-95%; and glycerin can range from 1-10%. The optimal solvent ratio for extraction will depend on the specific batch of Sarracenia purpurea and whether interbred genetic varieties were used, or related species such as Sarracenia flava and Sarracenia leukophylla.

The ratio of solvent used for extraction will also depend on the application. For example, higher concentrations of alcohol can cause a burning sensation when applied to open wounds, so an extract with less alcohol content may be used in patients with large open wounds, which can manifest from viral infections (such as Kaposi's Sarcoma).

The plant material must be weighed down during the extraction with a press, since the leaves tend to absorb the solvent. A ratio of 1:2 (plant material:solvent) is efficient for extracting the active components of Sarracenia purpurea; however, ratios that vary within 1:3, 1:4, 1:5, 1:6, 1:7, 1:8, 1:9, 1:10, 1:11, 1:12, 1:13, 1:14, 1:15, and 1:16 can be used for extraction depending on the desired potency of the extract. In other words, as the ratio increases, the more dilute the extract will be. During the extraction process, the Sarracenia purpurea absorbs over 50% of the solvent, and this amount increases with incubation time if the soak and press method is used. This can affect the final concentration of the extracted products; therefore, adjustments in the solvent ratio and incubation times must be optimized for each batch of plant material.

After the plant material is soaked for four weeks or longer, and the solvent is agitated daily, the liquid is decanted using a cheesecloth. The extract is stored in an amber glass bottle. The remaining plant material is pressed with the cheesecloth to expel any remaining liquid and the remaining plant material is composted. It is critical to store the extract in amber colored jars or clear jars away from sunlight in order to preserve the activity of the product. According to U.S. Pharmacopeia standards, the extract should be active for two months under these storage conditions (Green, James. The Herbal Medicine-Makers' Handbook. 2000).

The Coldfinger Extraction method is the preferred method of extraction for the purposes of this document and for the final product. The Coldfinger Extraction method is a proprietary extraction process developed by Eden Labs (Columbus, Ohio). Dr. Gowey purchased a Coldfinger Extractor (the Professional Model) to make the S. purpurea liquid extract. The Coldfinger Extraction method combines traditional soxhlet solvent distillation and steam distillation. This method enables the distillation to occur at lower temperatures, and the solvent (menstrum) that was used to extract the plant material can be recovered and recycled. There is an inverted condenser within an enclosed flask. This condenser points downwards into the flask. There is a soxhlet basket below the condenser. The condenser has cold liquid circulating through it to keep it cold during the course of the extraction. The solvent is placed at the bottom of the main flask. A ratio of organic alcohol to distilled water of 60:40 of 190 proof alcohol to distilled water works best for extraction depending on the inherent water content of the starting plant material. A 1:2 ratio of solvent:plant material is efficient for preparing the Sarracenia purpurea extraction; however, this ratio can be adjusted to variants of 1:3, 1:4, 1:5, 1:6, 1:7, 1:8, 1:9, 1:10, 1:11, 1:12, 1:13, 1:14, 1:15, and 1:16 if necessary.

The fresh, ground Sarracenia purpurea leaves are placed in the soxhlet basket, which has perforated sides and bottom to allow liquid to leave the basket. The basket is 18″*18″ and holds approximately 40-50 lbs. When the main flask is gently heated, the solvent begins to evaporate. The solvent vapors reach the cold condenser at the top of the flask and liquefy on the sides of the condenser. The condensed solvent flows down the sides of the condenser and drips off at the drip points on the end of the condenser. These drip points direct the solvent to flow into the soxhlet basket so it can saturate the ground Sarracenia purpurea. The solvent flows through the soxhlet basket and exits through the holes in the bottom of the basket. The solvent, which contains the Sarracenia purpurea extract, collects at the bottom of the flask. The initial solvent is dark in color because it contains high levels of Sarracenia purpurea extract. As the process continues, the solvent becomes clearer, indicating that the extraction is complete.

The solvent containing the extract is removed from the main flask. Glycerin may be added at a ratio of Alcohol:Water:Glycerin at 60:35:5 or 60:30:10 depending on the ratio of organic alcohol:distilled water used for extraction, which was dependent on the inherent water content of the batch of Sarracenia purpure; or the extract may be left at 60/40-65/35. The glycerin functions as a preservative of the Sarracenia purpurea extract. This extract can be directly used in a topical or suppository formulation. Alternatively, the solvent can be removed from the extract using a recovery vessel. The recovery vessel is a cup that is suspended below the condenser. The solvent containing the extract is heated, and the vapors re-condense on the condenser. The vapors drip off the tip of the condenser and fall into the cup; thus, they are separated from the extract. At the end of this process, a paste containing the extract is left behind. The remaining plant material in the soxhlet basket can be squeezed to remove any remaining solvent. The recovered solvent can be used to prepare an extract from a fresh batch of Sarracenia purpurea.

A vacuum can be applied during the extraction process. Applying a vacuum to the inside of the main flask lowers the boiling point of the solvent, enabling the operator to distill the solvent much more rapidly and at a lower temperature. When the vacuum is applied, solvent vapors migrate out of the port through which the vacuum is being pulled. A cold trap recondenses the solvent vapors and sends the liquid solvent back into the main flask. The extraction process can proceed for 1-3 days, and it is theoretically possible to collect 30 gallons of very concentrated, high-quality liquid extract.

Glycerin is added to the liquid extract at a ratio of Alcohol:Water:Glycerin at 60:35:5 or 60:30:10 depending on the ratio of organic alcohol:distilled water used for extraction, which was dependent on the inherent water content of the batch of Sarracenia purpurea. The addition of glycerin preserves the extract and improves its taste. Glycerin is not a menstrum, so it must be added at the very end of the extraction process.

The Coldfinger Extraction method generates a more concentrated extract than the soak and press technique because of the solvent and steam distillation processes, so the Coldfinger Extraction method is the preferred method of extraction, and will always be used to make the S. puruprea extracts used in topical formulations for the present composition. In addition, this method is more amenable to large-scale production than the soak and press technique, so it can be utilized when large amounts of Sarracenia purpurea extract are required. The coldfinger method also keeps leaf detritus out of the ending liquid extract.

The Sarracenia extract can be formulated into a topical gel (herein referred to as “Gowey Protocol Gel”. An extract of Sarracenia purpurea using a solvent ratio of 60:40 or 65/35 190 proof alcohol (such as Everclear or grain alcohol) to distilled water works well to mix in with the base of the Gowey Protocol Gel, the versabase gel. Versabase is manufactured and patented by the Professional Compounding Center of America. It contains ammonium acryloyldimethyltaurate/VP Copolymer, aloe vera, edetate disodium, allantoin, and methylchloroisothiazolinone/methylisothiazolinone. 20 mL aliquot of the S. purpurea extract (along with 0.01-2.0% liquid or powder anthocyanins) is slowly added to 100 gm of Versabase gel while stirring. The mixture is mixed for 1-30 s using an electronic mortar and pestle. The mixture is dispensed in a light-resistant 60 gm ointment tube. This Versabase gel mixture has an expiration up to one year.

In some embodiments, an analgesic, e.g., Saparin, may be used in conjunction with or as a substitution for the Pitcher Plant extract. See U.S. Pat. No. 7,597,687 (issued to Pauza on Oct. 6, 2009), the disclosure of which is incorporated in its entirety by reference herein.

The Sarracenia purpurea extract can be formulated as a suppository. For each suppository, 0.0935 gm Sarracenia purpurea tincture, 0.02 gm silica gel, 1.73 gm PCCA MBK™ fatty acid, and 0.0935 gm polysorbate 80 NF are required. When preparing n suppositories, always prepare enough reagents for n+1. The Sarracenia purpurea extract and other powder ingredients are placed in molds. The blue or small shell mold is used for weights <300 mg. The pink or large shell molds are used for weights >300 mg. The total weight of all the powder ingredients is determined, including the silica gel, and this value is multiplied by 70%. This value is subtracted from the blank weight of the suppository (Base PCCA MBK™ is 1.87 gm/medium shell mold). This value is the weight of PCCA MBK™ fatty acid that should be used per suppository. The PCCA MBK™ fatty acid is melted at 50° C. The Sarracenia purpurea tincture and silica gel are triturated into a fine powder. The powder is sifted into the molten PCCA MBK™ fatty acid while stirring. A strainer (PCCA #35-1414/#35-1896) is used. The heat is turned off, and the mixture is stirred until the powder is suspended. The mixture is poured into molds and allowed to cool at room temperature.

In some embodiments, the fatty acid components comprises one or more of the following ingredients: olive oil, flax seed oil, jojoba oil, cocoa butter, lecithin, castor oil, magnesium oil, apricot seed oil, rose seed oil, beeswax, palm oil, soybean oil, canola oil, safflower oil, peanut oil, grapeseed oil, sesame oil, rice bran oil, and other vegetable oils.

Additional homeopathic compounds can be added to the Sarracenia purpurea topical or suppository formulation.

The Gowey Protocol Gel is applied directly to the diseased tissue. In the case of cervical lesions induced by HPV infection, a 4 gram vaginal applicator is used by the patient to apply the topical gel formulation, while a cervical brush is used to apply the gel to the cervix directly by the physician (once a month) at follow-up visits. The vaginal applicator is filled inside with one to four grams (this is an individual prescription based on the amount of gel each patient's vaginal canal may fit) of the Gowey Protocol gel extract. The outer surface of the applicator can be covered with pure aloe gel in order to ease its insertion into the vaginal cavity. The treatment should be applied nightly or in the very least, twice weekly (depending on the individual patient prescription). It is important to maximize the contact of the gel with dysplastic areas. The applicator is washed with soap and water and then stored for future applications. This process is repeated daily until the tissues appear healthy (free of discoloration or discharge, which varies from patient to patient but the inventor has seen, in general, to be within 6 months). PCCA mixes tubes of the Gowey Protocol Gel in 30 or 60 grams. (PCCA is Professional Compounding Centers of America, located at 9901 South Wilcrest Dr., Houston, Tex. 77099-5132).

During the treatment, the intake of vegetables, fruits, protein-rich foods (such as meats, nuts, and beans), and rice is increased while common food sensitivity-causing foods such as wheat, dairy, sugars, and processed foods is eliminated during treatment. Multivitamin capsules (by Integrative Therapeutics) with high levels of B vitamins are taken three-four times daily during the treatment course in addition to bi-weekly intramuscular B vitamin of folic acid/B6 injections (50:50 ratio, which can be adjusted depending on the individual needs of the patient) or monthly/bimonthly intravenous vitamin therapy containing high levels of folic acid. Folic acid deficiency has been linked to cervical dysplasia; the inventor has also noted that patients with diets high in alcohol, sugar, food allergens, who use birth control pills, or who are exposed to high levels of environmental toxins such as solvents and heavy metals, do not respond as quickly to the Gowey Protocol gel, thereby making it essential to remove some of these factors from the patient lifestyle/diet. Stress reduction management techniques should be implemented, such as exercise, spending time with family, deep breathing, or partaking in hobbies. Stress induces nutrient depletion as does the use of alcohol, consumption of sugar/processed foods, and use of birth control. It is imperative that the patient utilizes protection measures when partaking in sexual activities during their treatment course in order to avoid acquiring additional HPV subtypes. However, patients who are monogamous have used the Gowey Protocol gel without protection, as a lubricant.

### EXAMPLES

The following are non-limiting examples of the various applications of the present composition:

Case one: Patients presented with cervical dysplasia. Inventor topically applied the present compositions to the affected region; patient then inserted the gel vaginally via a vaginal applicator (at night) from two to seven night a week. Patients experienced complete reversal of symptoms from ASGUS (abnormal cells), LSIL (low grade dysplasia), and HSIL (high grade dysplasia) to normal, within 6 months. 6 months is the standard of care for re-paping patients (when they have had an abnormal pap). Paps were obtained at 3 and 6 months to monitor progress. Some patients had a period of dark discharge from the cervix after the first application of the Gowey Protocol Gel, and when the bleeding/discharge stopped dramatic shift in the appearance of the cervical tissues is visible (from red and angry looking to pink, which is the normal appearance).

The optimal method to screen for cervical pre-cancerous and cancerous changes, which often result from HPV infections, is the ThinPrep Papanicolaou test. This test detects premalignant and malignant cells in the endocervical canal (transformation zone). Cells are collected from the outer opening of the cervix and examined under the microscope to look for abnormalities. All the patients who participated in this case trial underwent a pre-treatment and post-treatment ThinPrep Papanicolaou test, which was administered and evaluated by a private diagnostic laboratory. Prior to treatment, this patient was diagnosed as having an epithelial cell abnormality; there were atypical squamous cells of undetermined significance. This diagnosis indicates a 50% probability of having a squamous intraepithelial lesion on a directed biopsy. When a pre-cancerous or cancerous cervical lesion is identified, this suggests that the patient is infected with a high-risk HPV type. In addition to cytology, the ThinPrep Papanicolaou test also detects infection with high-risk HPV types by assessing whether HPV DNA is present in the cervical sample. In the provided example, the patient was determined to be infected with high-risk HPV (types 16, 18, 31, 33, 35, 39, 45, 51, 52, 56, 58, 59, or 68) prior to treatment.

The post-treatment ThinPrep Papanicolaou test results for this patient indicate that there are no intraepithelial lesions or malignancies present. Therefore, these cytology results confirm that treatment with the Sarracenia purpurea extract resulted in a reduction in the physical manifestation of HPV disease when applied over time (different for each patient but tends to be within 1-6 months). The post-treatment ThinPrep Papanicolaou test results also indicate that there is no detection of HPV DNA, so the HPV infection appears to have been eradicated by the Sarracenia purpurea extract treatment. Therefore, the Sarracenia purpurea extract exhibits potent anti-viral and anti-cancer activities against HPV, which confirms the apoptosis activities of anthycyanadins.

Case two: Patient presented with herpes simplex I or II during an office visit. Dr. Gowey topically applied the present composition to the affected region. Patient experienced immediate relief of pain; patient continued to apply the Gowey Protocol to the lesions every 3-4 hours, as prescribed. Lesions were gone, according to the patient, or crusting over within two days.

Case three: Clinical trial with herpes simplex I or II patients. There were 33 patients, including both HSV I and II subtypes, in the study. The patients were treated daily with the topical Sarracenia purpurea extract versabase formulation (Gowey Protocol Gel) or a placebo for 14 days. The assignment of patients to each group was random. The number of lesions was determined on days 1, 3, 5, and 14. On day 1, the patients receiving the Sarracenia purpurea extract had 25 lesions and those patients receiving placebo had 13 lesions. On day 3, the patients receiving the Sarracenia purpurea extract still had 25 lesions and those patients receiving placebo had 15 lesions, indicating disease progression. On day 5, there was a reduction in the number of lesions for the Sarracenia purpurea extract group; the number of lesions decreased from 25 to 10. There was a decrease of only one lesion for the placebo group, and this reduction was not sustained at day 14. There was a further reduction in the number of lesions for the Sarracenia purpurea extract group at day 14; the number of lesions was reduced to only one. Therefore, treatment with the Sarracenia purpurea extract resulted in a reduction in the number of HSV lesions, suggesting that the Sarracenia purpurea extract was efficient in reducing the physical manifestation of HSV disease when administered daily for 14 days.

The mean size of the lesions for each patient group was recorded during the course of the study. Both patients receiving the Sarracenia purpurea extract and placebo had similar-sized lesions of 8.2 and 8.3, respectively on day 1. On day 3, only the group receiving the Sarracenia purpurea extract experienced a reduction in the size of their lesions; there was a 50% reduction from 8.2 to 4.2 mm. (There was further reduction in the mean lesion size at day 5; it was reduced to 1.5 mm. There was only a small reduction in the mean size of the lesions for the placebo group; it decreased from 8.3 mm to 6.9 mm. By day 14, the one lesion remaining for the Sarracenia purpurea extract group was only 0.2 mm, whereas the mean size of the lesions for the 15 remaining lesions for the placebo group was 5.4 mm. Therefore, treatment with the Sarracenia purpurea extract resulted in a reduction in the size of the HSV lesions, suggesting that the Sarracenia purpurea extract was efficient in reducing the physical manifestation of HSV disease when administered daily for 14 days.

The pain scale self-reported by patients was also assessed during the study. The mean pain scale value for patients receiving the Sarracenia purpurea extract was 6.1 on day 1, and the mean pain scale value for patients receiving the placebo was 4.1 on day 1. The mean pain scale value significantly decreased for patients receiving the Sarracenia purpurea extract by day 1; it was only 0.2. This decrease in the mean pain scale value decreased to 0.0 by day 5, and this was reduction was sustained at day 14. The mean pain scale value for patients receiving the placebo increased on days 3 and 5 to 4.7 and 4.6, respectively; however, it decreased to 1.7 by day 14. Therefore, treatment with the Sarracenia purpurea extract reduced the pain associated with HSV disease when administered daily for 14 days.

Case four: Patient presented with squamous cell carcinoma. Pre biopsy reveals squamous cell carcinoma, a type of skin cancer that requires a biopsy procedure as treatment called “Mohs technique”. This cancer can be very aggressive. Patient topically applied the present composition to the affected region while patient waited for their Moh's appointment with their dermatologist. However, follow up visit by patient to their dermatologist revealed post biopsy with normal tissues, therefore the Moh's was not performed.

Case five: Patient presented with VIN I, which is a type of vulvar cancer. Patient had had this for several years, a slowly growing lesion of 2.5 cm in diameter and a much different color than the normal tissues. Patient topically applied the present composition to the affected region. Tissues slowly healed after application of the present composition.

Case six: Patient presented with plantar/palmar warts. Patient topically applied the present composition to the affected region every 3-4 hours and kept the warts covered with a bandage. The topical application began to create changes to the wart within 5-7 days. Wart began to crust over and eventually fell off. Lesions were gone within 1-2 months.

Case seven: Patient presented with Kaposi's sarcoma. Patient topically applied the present composition to the affected region. The topical application prevented lesions from developing (normally the lesions ulcerate down to the bone).

Case eight: Five year old patient presented with MRSA. Was on antibiotics but they were not healing the lesions; had MRSA on his hands and feet, and tended to get this manifestation every few months. Dr. Gowey instructed patient's mother to apply the present composistion to the lesions every 3-4 hours. Lesions were gone within 24 hours and have not since returned.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A composition comprising a pitcher plant component and a fatty acid component.

2. The composition of claim 1 wherein the pitcher plant component is an extract.

3. The composition of claim 2 wherein the extract is an alcohol extract.

4. The composition of claim 1 wherein the pitcher plant component further comprises a preservative component.

5. The composition of claim 1 wherein the pitcher plant component is prepared through a soak and press technique.

6. The composition of claim 1 wherein the pitcher plant component is prepared through steam distillation.

7. The composition of claim 1 wherein the fatty acid component comprises a versabase ingredients.

8. The composition of claim 1 being a topical formulation.

9. The composition of claim 1 being a suppository formulation.

10. The composition of claim 1 wherein the ratio of the pitcher plant component to the fatty acid component (ml:mg) is about 20:100.

11. The composition of claim 1 wherein the ratio of the pitcher plant component to the fatty acid component (ml:mg) is about 10:100.

12. The composition of claim 1 wherein the ratio of the pitcher plant component to the fatty acid component (ml:mg) is about 5:100.

13. The composition of claim 1 wherein the ratio of the pitcher plant component to the fatty acid component (ml:mg) is about 30:100.

14. The composition of claim 1 wherein the ratio of the pitcher plant component to the fatty acid component (ml:mg) is about 50:100.

15. The composition of claim 1 wherein the ratio of the pitcher plant component to the fatty acid component (ml:mg) is about 20:110.

16. The composition of claim 1 wherein the fatty acid component comprises one or more of the following fatty acids, vegetable or cosmetic oils or bases used in topical preparations.

17. A method of making a topical formulation, the method comprising:
(a) obtaining a pitcher plant component;
(b) obtaining a fatty acid component; and
(c) mixing the pitcher plant component with the fatty acid component.

18. The method of claim 17 wherein the pitcher plant component is obtained via soak and press or steam distillation.

19. The method of claim 17 wherein the fatty acid component comprises the versa base formulation.

20. The method of claim 17 wherein the mixing comprises subjecting the pitcher plant component and fatty acid component to agitation with the mortar and pestle

21. The method of claim 17 further comprising storing the topical formulation in a light-resistant container.

22. A method of making a suppository formulation, the method comprising:
(a) obtaining a pitcher plant component
(b) obtaining a fatty acid component
(c) weighing the components
(d) mixing the pitcher plant component with the fatty acid component

23. The method of claim 22 wherein the pitcher plant component is obtained via soak and press or steam distillation.

24. The method of claim 22 wherein the fatty acid component comprises ammonium acryloyldimethyltaurate/VP copolymer

25. The composition of claim 24 wherein the fatty acid component further comprises methylchloroisothiazolinone/methylisothiazolinone.

26. The method of claim 22 wherein the mixing comprises adding the pitcher plant component to molten fatty acid component.

27. The method of claim 26 wherein the pitcher plant component is a fine powder.

28. The method of claim 27 wherein the fine powder comprises of pitcher plant extract and silica gel.

29. The method of claim 22 further comprising storing the suppository formulation in a light-resistant container.

30. A method of alleviating a cancerous lesion, the method comprising:
(a) obtaining a topical formulation which comprises a pitcher plant component and fatty acid component; and
(b) administering the formulation to the cancerous lesion.

31. The method of claim 30 further comprises administering vitamin B.

32. The method of claim 30 further comprises administering folic acid.

33. The method of claim 30 further comprises administering vitamin B and folic acid.

34. The method of claim 30 further comprises administering homeopathic therapy as needed on an individual level and basis per patient, in a manner that is determined appropriate by the physician.
